(19) United States
(12) Patent Application Publication
(10) Pub. No.: US 2016/0377605 A1
(43) Pub. Date: Dec. 29, 2016

(54) LYSOSOMAL ATP SELECTIVE TWO-PHOTON ABSORBING FLUORESCENT PROBE

(71) Applicant: POSTECH ACADEMY-INDUSTRY FOUNDATION, Pohang-si (KR)

(72) Inventors: Kyo Han AHN, Pohang-si (KR); Yong Woong JUN, Pohang-si (KR); Donghee MA, Pohang-si (KR)

(73) Assignee: POSTECH ACADEMY-INDUSTRY FOUNDATION, Pohang-si (KR)

(21) Appl. No.: 15/194,188

(22) Filed: Jun. 27, 2016

(30) Foreign Application Priority Data
Jun. 26, 2015 (KR) 10-2015-0091307

(51) Int. Cl. G01N 33/53; G01N 33/58; G01N 21/64; C07F 5/02

(52) U.S. Cl. CPC G01N 33/5308; C07F 5/022; G01N 33/582; G01N 21/6428; G01N 2400/00; G01N 2021/6439

(57) ABSTRACT

A fluorescent probe compound, a preparation method thereof, and a method of imaging and quantifying lysosomal adenosine triphosphate (ATP) at a cell or tissue level through one-photon or two-photon fluorescence microscopy using the compound is disclosed. A fluorescence detection system capable of detecting lysosomal ATP is further disclosed. Since the fluorescent probe compound is found to be capable of selectively sensing and quantifying lysosomal ATP in a cell or tissue, it is expected that the disclosed compound or composition can be usefully employed in the study of various biological reactions or diseases associated with ATP in a living body.

### CROSS-REFERENCE TO RELATED APPLICATION

This application claims priority to and the benefit of Korean Patent Application No. 10-2015-0091307, filed on Jun. 26, 2015, the disclosure of which is incorporated herein by reference in its entirety.

The present invention was made with the support of the Ministry of Science, ICT and Future Planning of Republic of Korea for Project #2014064569 of the Global Research Lab (GRL) Program and Project #2014028940 of the Advanced Research Center Program.

### BACKGROUND

1. Field

The present disclosure relates to a novel fluorescent probe for selectively detecting adenosine triphosphate (ATP) in lysosomes and quantifying the analyte using a ratio between two types of fluorescence emitted upon detection, a preparation method thereof, a composition for ATP detection comprising the fluorescent probe, an ATP detection method using the composition, a method of imaging a cell or a tissue, and a quantification method of ATP.

2. Discussion of Related Technology

Organic phosphate anions such as adenosine triphosphate (ATP), adenosine diphosphate (ADP), and adenosine monophosphate (AMP) are substances of much academic interest because of their variety of biochemical activities.

Among them, ATP is an important substance that is involved in various reaction mechanisms in vivo as a main energy transferring substance and a signal transferring substance and is recognized as the “molecular unit of currency” in a living body. According to recent studies, a rich amount of ATP is comprised in the lysosome, which is a cell organelle, and ATP released extracellularly by the exocytosis of the lysosome plays an essential role in signaling immunogenic cell death, apoptosis, and neurotransmission.

### SUMMARY

The present inventors enabled the imaging of a cell or a tissue through one-photon or two-photon fluorescence microscopy by developing a novel fluorescent probe for the selective detection of adenosine triphosphate (ATP) in lysosomes and designing the fluorescence detection system to have a two-photon absorption characteristic.

In addition, the present inventors realized a ratiometric system for sensing an analyte to develop a method capable of quantifying an analyte in various conditions, and thereby completed the present invention. The ratiometric system is a detection system capable of quantifying an analyte under in vivo conditions in which the prediction of the environment is impossible, by relying upon the fact that, despite the sensitivity of individual fluorescence intensity to various environmental factors, the ratio between fluorescence intensities is not sensitive to changes in the environment.

Therefore, the present application is directed to providing a novel fluorescent probe compound, a preparation method thereof, a composition for ATP detection comprising the fluorescent probe compound or a pharmaceutically acceptable salt of the compound, an ATP detection method using the compound or the pharmaceutically acceptable salt of the compound, and a method of imaging and quantifying the lysosomal ATP at a cell or tissue level.

However, the aspects of the present invention are not limited to those mentioned above, and other aspects not addressed herein will be clearly understood by those skilled in the art from the following descriptions.

One aspect of the present invention provides a compound represented by Structural Formula 1 below:

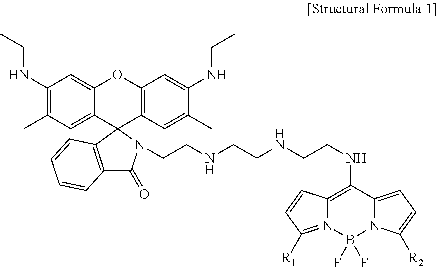

In the Structural Formula 1 above, R₁ and R₂ are each independently hydrogen (H), a methyl group (Me), an allyl group, or a C₂ to C₁₂ alkyl group.

In one embodiment of the present invention, the compound represented by the Structural Formula 1 is 10-((2-((2-((2-(3′,6′-bis(ethylamino)-2′,7′-dimethyl-3-oxospiro[isoindoline-1,9′-xanthen]-2-yl)ethyl)amino)ethyl)amino)ethyl)amino)-5,5-difluoro-5H-dipyrrolo[1,2-c:2′,1′-f][1,3,2]diazaborinin-4-ium-5-uide.

In addition, another aspect of the present invention provides a method of detecting ATP, wherein the method comprises a process of treating a cell or a tissue with a composition comprising the compound represented by the Structural Formula 1 or a pharmaceutically acceptable salt of the compound.

In one embodiment of the present invention, the method selectively detects lysosomal ATP.

In another embodiment of the present invention, the composition binds with ATP to enhance the fluorescence intensity within the range of 520 to 580 nm.

In still another embodiment of the present invention, the method detects ATP in the pH range of 4.5 to 6.

In addition, still another aspect of the present invention provides a method of preparing the compound represented by the following Structural Formula 1, wherein the method comprises the processes described below:

a) adding triethylene tetramine to rhodamine 6G ((E)-ethyl 2-(6-(ethylamino)-3-(ethylimino)-2,7-dimethyl-3H-xanthene-9-yl)benzoate) to synthesize 2-(2-((2-((2-aminoethyl)amino)ethyl)amino)ethyl)-3′,6′-bis(ethylamino)-2′,7′-dimethylspiro[isoindoline-1,9′-xanthen]-3-one, which is a rhodamine 6G derivative compound; and

b) reacting the 2-(2-((2-((2-aminoethyl)amino)ethyl)amino)ethyl)-3′,6′-bis(ethylamino)-2′,7′-dimethylspiro[isoindoline-1,9′-xanthen]-3-one with a compound represented by the following Structural Formula 2 in dichloromethane (DCM) to synthesize the compound represented by the following Structural Formula 1.

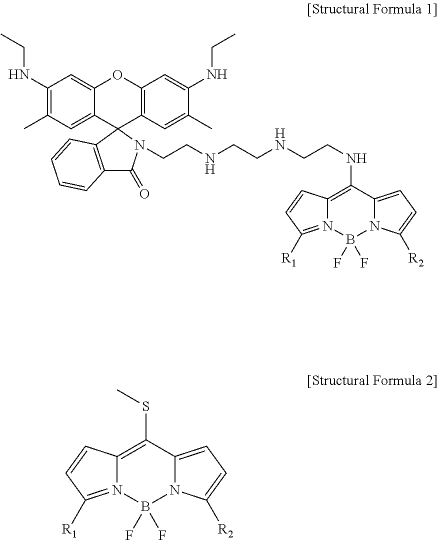

In addition, yet another aspect of the present invention provides a method of imaging a cell or a tissue, wherein the method comprises the processes described below:

a) treating a cell or a tissue with the compound represented by Structural Formula 1 or a pharmaceutically acceptable salt thereof; and

b) observing, through one-photon or two-photon fluorescence microscopy, the fluorescence emitted from the cell or tissue due to lysosomal ATP.

In addition, a further aspect of the present invention provides a method of quantifying lysosomal ATP, wherein the method comprises the processes described below:

a) treating a cell or tissue with the compound represented by Structural Formula 1 or a pharmaceutically acceptable salt thereof;

b) measuring a fluorescence intensity from the cell or tissue at wavelengths ranging from 440 to 460 nm and from 520 to 580 nm; and

c) quantifying ATP concentration by calculating a ratio between the two measured fluorescence intensities.

Having a two-photon absorption characteristic, the fluorescent probe according to embodiments of the present invention for selectively sensing lysosomal ATP enables the selective detection and imaging of lysosomal ATP in a cell and a deep tissue by one-photon or two-photon microscopy. In addition, such a probe exhibits a ratiometric characteristic in sensing ATP, and thus, enables the quantification of ATP even when little is known about the environment. Therefore, the high-resolution imaging and quantification of lysosomal ATP, which is a main biological substance and was previously not sensed in a selective manner, is possible, and thus, it is expected that the fluorescent probe according to embodiments of the present invention will be applicable to the studies of various biochemical reactions involving ATP.

### BRIEF DESCRIPTION OF THE DRAWINGS

The above and other features and advantages of the present invention will become more apparent to those of ordinary skill in the art by describing in detail embodiments thereof with reference to the accompanying drawings, in which:

FIG. 1A and FIG. 1B are the results of observing an absorption characteristic of Compound 4 (R₁═R₂═H), which is a fluorescent probe. FIG. 1A is a result of measuring an absorbance spectrum in a pH-5.5 PBS solution (with 0.1% acetonitrile) comprising the Compound 4 at the concentration of 10 μM, and FIG. 1B is a result of measuring an absorbance spectrum in a pH-5.5 PBS solution (with 0.1% acetonitrile) comprising the Compound 4 at the concentration of 10 μM and ATP at 1 mM;

FIG. 2A and FIG. 2B are the results of observing a fluorescence characteristic of Compound 4 (R₁═R₂═H), which is a fluorescent probe. FIG. 2A is a result of measuring a fluorescence spectrum in a pH-5.5 PBS solution (with 0.1% acetonitrile) comprising the Compound 4 at the concentration of 10 μM and ATP (0.0-1.0 mM), and FIG. 2B is a result of verifying, through the correlation between the ATP concentration (x-axis) and the ratio between two types of fluorescence (y-axis), the possibility of analyte quantification through a ratiometric detection system;

FIG. 3A is a result of measuring a fluorescence spectrum in a PBS solution (with 0.1% acetonitrile) with pH of 4.0 to 8.0 comprising the Compound 4 at the concentration of 10 μM and ATP (0.0-1.0 mM) to verify the correlation between the ATP detection by the Compound 4 (R₁═R₂═H) and the acidity of the solution, and FIG. 3B is a result of measuring changes in the ratio of two types of fluorescence (y-axis) with respect to the ATP concentration (x-axis) in the pH range of 4.5 to 5.5;

FIG. 4 is a result of measuring the fluorescence spectra of the Compound 4 (R₁═R₂═H) at the concentration of 10 μM and various cations and anions to verify the selectivity of the Compound 4 in ATP detection;

FIG. 5 is a result of determining the cytotoxicity of the Compound 4 (R₁═R₂═H) by treating HeLa cells with the compound;

FIG. 6 is a result of observing a phenomenon of fluorescence emission by one-photon and two-photon fluorescence microscopy to verify the lysosomal selectivity of the Compound 4 (R₁═R₂═H), which is a fluorescent probe;

FIG. 7A and FIG. 7B are the results of verifying the lysosomal selectivity of the Compound 4 (R₁═R₂═H), which is a fluorescent probe. FIG. 7A is a result of comparing fluorescence patterns within a selected region, which is represented as a position in the region (x-axis) versus the fluorescence intensity (y-axis), and FIG. 7B is a result of determining the colocalization factor of the intensities of two types of fluorescence in FIG. 7A; and

FIG. 8 is a result of observing, by two-photon fluorescence microscopy, the changes in fluorescence after treating a rat brain tissue with the Compound 4 (R₁═R₂═H), which is a fluorescent probe.

### DETAILED DESCRIPTION OF EMBODIMENTS

As discussed above, a rich amount of ATP is included in the lysosome, which is a cell organelle, and ATP released extracellularly by the exocytosis of the lysosome plays an essential role in signaling immunogenic cell death, apoptosis, and neurotransmission. However, due to the current non-existence of a detection system capable of selectively detecting and quantifying only the ATP in lysosomes, there is much difficulty in studying the aforementioned reaction mechanisms.

Among ATP probes, the probes based on changes in fluorescence are useful detection systems that have high sensitivity, selectivity, spatiotemporal resolution, noninvasiveness, and biocompatibility and deliver information in a living body in the form of direct visual information through visual bioimaging.

Among the fluorescent probes, those including a two-photon absorbing material can be used with two-photon microscopy. Using photons with longer wavelength and lower energy compared to confocal microscopy and one-photon microscopy, two-photon microscopy has characteristics appropriate for visual bioimaging due to a large penetrability (>500 μm) and the ability to minimize tissue autofluorescence and self-absorption, produce high resolution images, and reduce photodamage and photobleaching. However, since the fluorescence intensity itself easily changes with the solvent, device, concentration, environment (pH, viscosity, the degree of polarity, etc.) and the like, the probes based on changes in fluorescence cannot quantify analytes.

Therefore, there is a need for the development of a ratiometric system that emits two types of fluorescence when one detection system detects an analyte, where the ratio between the types of fluorescence gradually changes with the concentration of the analyte, but the development of such system is incomplete up to date.

One aspect of the present invention provides a compound represented by the following Structural Formula 1, which is a novel fluorescent probe.

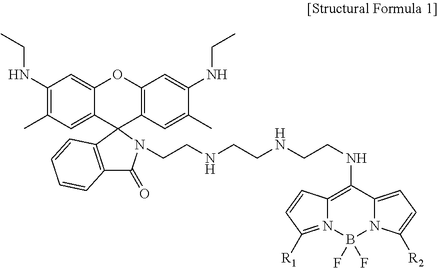

In the Structural Formula 1 above, R₁ and R₂ are each independently hydrogen (H), a methyl group (Me), an allyl group, or a C₂-C₁₂ alkyl group.

In embodiments, the R₁ and R₂ of Structural Formula 1 are hydrogen, and the compound is represented by the following Structural Formula 3, but is not limited thereto.

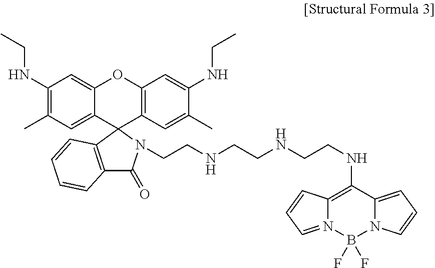

In one embodiment of the present invention, the feasibility of the quantification of adenosine triphosphate (ATP) based on a fluorescence characteristic outside a body and the substantial selective detection of lysosomal ATP at a cell or tissue level by applying the quantification method to a cell or a tissue was confirmed (Example 3 to Example 7).

Therefore, embodiments of the present invention may provide a method of detecting ATP, wherein the method comprises a process of treating a cell or a tissue with a composition comprising the compound represented by the Structural Formula 1 or a pharmaceutically acceptable salt of the compound.

Embodiments of the present invention may provide a method of imaging and quantifying lysosomal ATP in vivo using the compound according to embodiments of the present invention or a pharmaceutically acceptable salt thereof.

The method may be able to detect lysosomal ATP in a specific manner, but is not limited thereto.

In addition, by binding to ATP, the composition may be able to increase the fluorescence intensity in the range of 520 to 580 nm, and in embodiments, increases the fluorescence intensity in the range of 550 to 570 nm, but range is not limited to those provided herein.

Moreover, the method may be able to detect ATP in the pH range of 4.5 to 6, and in embodiments, my detect ATP in the pH range of 4.8 to 5.8, but the range is not limited to those provided herein.

Another aspect of the present invention may provide a method of preparing the compound represented by the following Structural Formula 1, wherein the method comprises the processes described below:

a) adding triethylene tetramine to rhodamine 6G ((E)-ethyl 2-(6-(ethylamino)-3-(ethylimino)-2,7-dimethyl-3H-xanthene-9-yl)benzoate) to synthesize 2-(2-((2-((2-aminoethyl)amino)ethyl)amino)ethyl)-3′,6′-bis(ethylamino)-2′,7′-dimethylspiro[isoindoline-1,9′-xanthen]-3-one, which is a rhodamine 6G derivative compound; and

b) reacting the 2-(2-((2-((2-aminoethyl)amino)ethyl)amino)ethyl)-3′,6′-bis(ethylamino)-2′,7′-dimethylspiro[isoindoline-1,9′-xanthen]-3-one with a compound represented by the following Structural Formula 2 in dichloromethane (DCM) to synthesize the compound represented by the following Structural Formula 1.

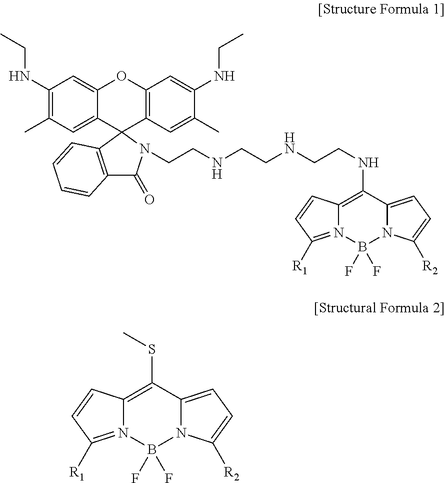

Still another aspect of the present invention may provide a method of imaging a cell or a tissue, wherein the method comprises the processes described below:

a) treating a cell or a tissue with the compound represented by Structural Formula 1 or a pharmaceutically acceptable salt thereof; and

b) observing, through one-photon or two-photon fluorescence microscopy, the fluorescence emitted from the cell or tissue due to lysosomal ATP.

Yet another aspect of the present invention may provide a method of quantifying lysosomal ATP, wherein the method comprises the processes described below:

a) treating a cell or tissue with the compound represented by Structural Formula 1 or a pharmaceutically acceptable salt thereof;

b) measuring a fluorescence intensity from the cell or tissue at wavelengths ranging from of 440 to 460 nm and from 520 to 580 nm; and

c) quantifying ATP concentration by calculating a ratio between the two measured fluorescence intensities.

Hereinafter, examples of the invention will be described for promoting an understanding of the invention. However, the following examples should be considered in a descriptive sense only and the scope of the invention is not limited to the following examples.

### EXAMPLE 1

### Synthesis of Compound 2; (2-(2-((2-((2-aminoethyl)amino)ethyl)amino)ethyl)-3′,6′-bis(ethylamino)-2′,7′-dimethylspiro[isoindoline-1,9′-xanthen]-3-one)

The general route of synthesis of Compound 2 is provided in the following Reaction 1.

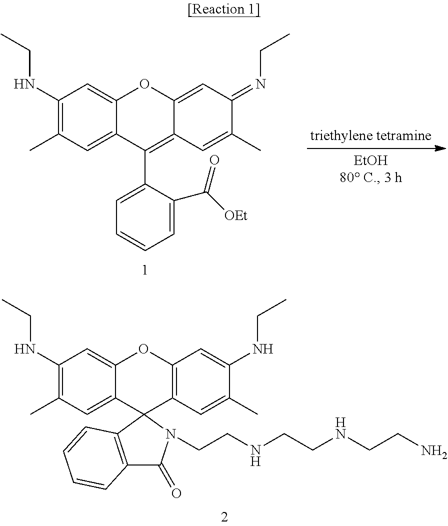

The present inventors carried out the synthesis of (2-(2-((2-((2-aminoethyl)amino)ethyl)amino)ethyl)-3′,6′-bis(ethylamino)-2′,7′-dimethylspiro[isoindoline-1,9′-xanthen]-3-one), which is Compound 2.

Compound 1 (rhodamine 6G, 100 mg, 0.23 mmol) ((E)-ethyl 2-(6-(ethylamino)-3-(ethylimino)-2,7-dimethyl-3H-xanthene-9-yl)benzoate), which is a synthetic starting material available on the market, was put in a 100 mL container and was dissolved in 20 mL ethanol. Triethylene tetramine (575 μL, 2.3 mmol) was added and a reflux condenser was connected to the container, and then the contents were subjected to reflux and stirring for 3 hours at 80° C. in a silicone oil bath. The temperature of the mixture was reduced to room temperature (25° C.), the solvent was removed therefrom under a reduced pressure at 40 mbar, and then the mixture was dissolved in a 30 mL saturated sodium bicarbonate (NaHCO₃) solution and was extracted twice with 20 mL dichloromethane (DCM). The extracted organic layer was washed with saturated brine (10 mL) and was dried with anhydrous sodium sulfate. Yellow solid Compound 2 (49.9 mg) was obtained by removing the solvent under a reduced pressure at 40 mbar and then purifying the resultant substances through silica gel (Merk-silicagel 60, 230-400 mesh) by column chromatography (with MeOH/DCM/TEA=20:80:1 as the developing solution), and was used for subsequent processes without further separation.

### EXAMPLE 2

### Synthesis of Compound 4 (R₁═R₂═H); (10-((2-((2-((2-(3′,6′-bis(ethylamino)-2′,7′-dimethyl-3-oxospiro[isoindoline-1,9′-xanthen]-2-yl)ethyl)amino)ethyl)amino)ethyl)amino)-5,5-difluoro-5H-dipyrrolo[1,2-c:2′,1′-f][1,3,2]diazaborinin-4-ium-5-uide)

The general route of synthesis of Compound 4 (R₁═R₂═H) is provided in the following Reaction 2.

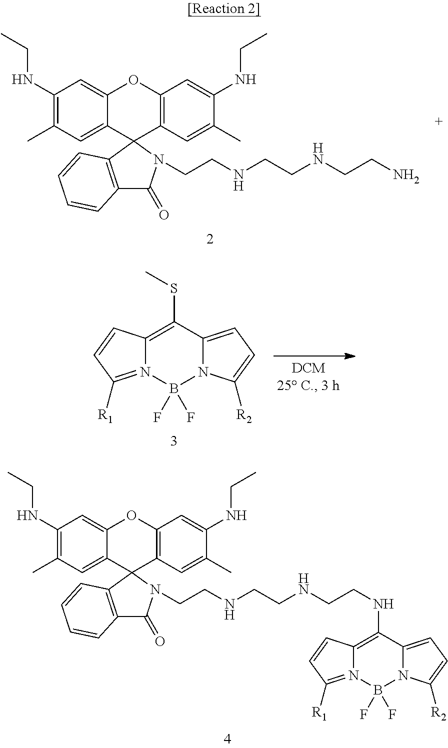

The present inventors carried out the synthesis of 10-((2-((2-((2-(3′,6′-bis(ethylamino)-2′,7′-dimethyl-3-oxospiro[isoindoline-1, 9′-xanthen]-2-yl)ethyl)amino)ethyl)amino)ethyl)amino)-5,5-difluoro-5H-dipyrrolo[1,2-c:2′,1′-f][1,3,2]diazaborinin-4-ium-5-uide, which is Compound 4 (R₁═R₂═H).

Specifically, the yellow solid Compound 2 (49.9 mg) obtained from Example 1 was put in a 25 mL container in an anhydrous argon atmosphere from which oxygen was removed, and the compound was dissolved in dry dichloromethane (4 mL). A known Compound 3 (8-(Thiomethyl)4,4-difluoro-4-bora-3a,4a-diaza-sindacene, C₁₀H₉BF₂N₂S) (20 mg, 0.084 mmol) was added in the container and the contents were stirred for 3 hours at room temperature (25° C.).

Light yellow solid Compound 4 (R₁═R₂═H) (54 mg, 32%) was obtained by removing the solvent from the mixture under a reduced pressure at 40 mbar, and purifying the resultant substances through silica gel (Merk-silicagel 60, 230-400 mesh) by column chromatography (with 5% MeOH/DCM as the developing solution).

¹H NMR (CDCl₃, 600 MHz, 296 K): d 7.90 (m, 1H), 7.73 (s, 1H), 7.48 (m, 3H), 7.18 (s, 1H), 7.08 (m, 2H), 6.56 (s, 1H), 6.37 (s, 2H), 6.34 (s, 1H), 6.25 (s, 2H), 3.78 (t, J=6 Hz, 2H), 3.54 (d, J=4.8 Hz, 2H), 3.30 (t, J=6 Hz, 2H), 3.22 (m, 4H), 3.07 (t, J=5.4 Hz, 2H), 2.63 (dd, J=16.8, 2.4 Hz, 4H), 2.41 (t, J=6 Hz, 2H), 2.04 (d, J=9 Hz, 2H), 1.92 (s, 6H), 1.34 (t, J=7.2 Hz, 6H); ¹³C NMR (CDCl₃, 600 MHz, 298 K): d 169.0, 153.6, 151.8 (2 carbons), 148.2, 147.5 (2 carbons), 135.1, 132.7, 132.0, 130.9, 128.3 (2 carbons), 128.2, 123.9 (2 carbons), 122.9, 122.8 (2 carbons), 118.0 (2 carbons), 115.1, 114.5, 113.4, 105.9, 95.6 (2 carbons), 65.3, 48.3, 48.0, 47.9, 46.2 (2 carbons), 45.0, 40.2, 38.4 (2 carbons), 16.7 (2 carbons), 14.7 (2 carbons); HRMS (FAB): m/z calcd. for C₄₁H₄₇BF₂N₈O₂, 732.67; found,733.40.

### EXAMPLE 3

Assessment of Absorption Characteristic of Fluorescent Probe

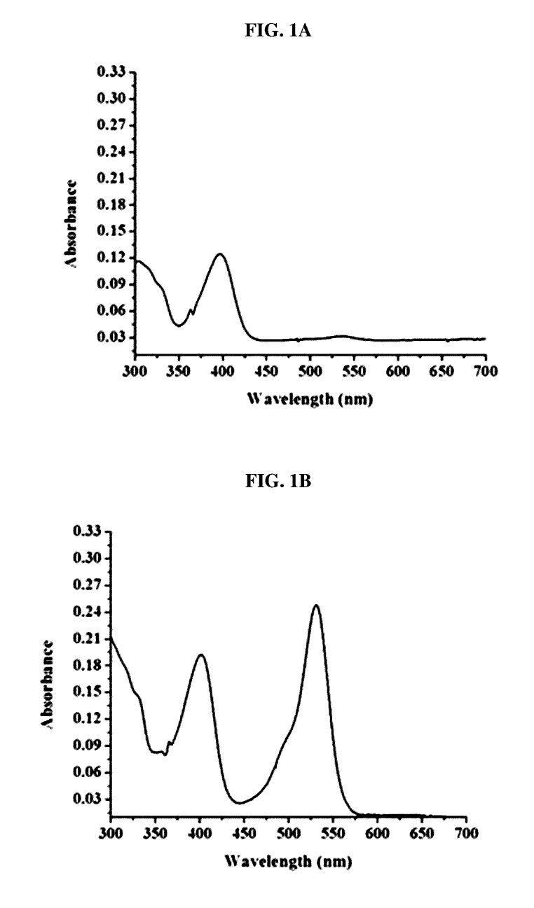

The present inventors observed the absorption characteristic of Compound 4 (R₁═R₂═H), which is the fluorescent probe according to embodiments of the present invention, and the results are provided in FIG. 1.

Specifically, to observe the absorption characteristic of the fluorescent probe, the present inventors measured an absorbance spectrum by filling, with a pH 5.5 PBS solution (with 0.1% acetonitrile) comprising 10 μM Compound 4, a quartz fluorescence cell (114F-QS, Hellma Analytics) with a 1 cm light path, and a graph of the absorbance (y-axis) with respect to the wavelength (x-axis) is shown in FIG. 1A. In addition, another absorbance spectrum was measured by filling a quartz fluorescence cell (114F-QS, Hellma Analytics) with a 1 cm light path with a pH 5.5 PBS solution (with 0.1% acetonitrile) comprising 10 μM Compound 4 and 1 mM ATP, and a graph of the absorbance (y-axis) with respect to the wavelength (x-axis) is shown in FIG. 1B. In FIG. 1A, the fluorescent probe had a single absorption near 400 nm. On the other hand, in FIG. 1B in which ATP was detected, a new absorption was observed near 540 nm in addition to 400 nm. The absorbance spectra were obtained using the HP 8453 UV/Vis spectrophotometer.

### EXAMPLE 4

Assessment of Fluorescence Characteristic of Fluorescent Probe

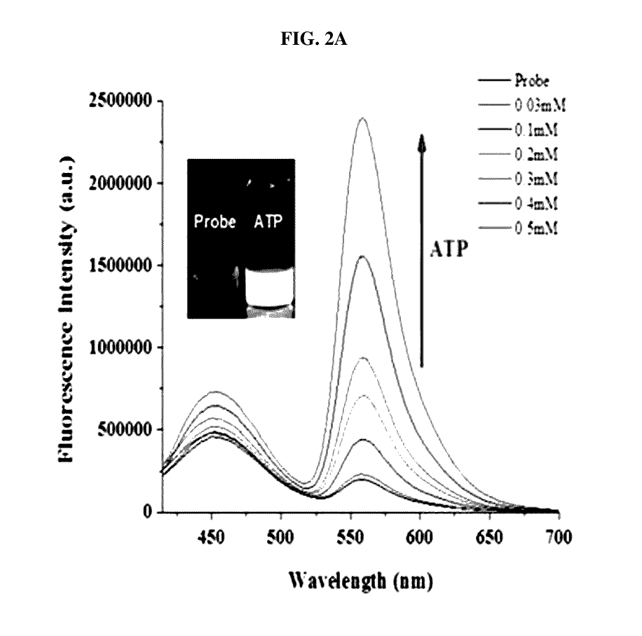

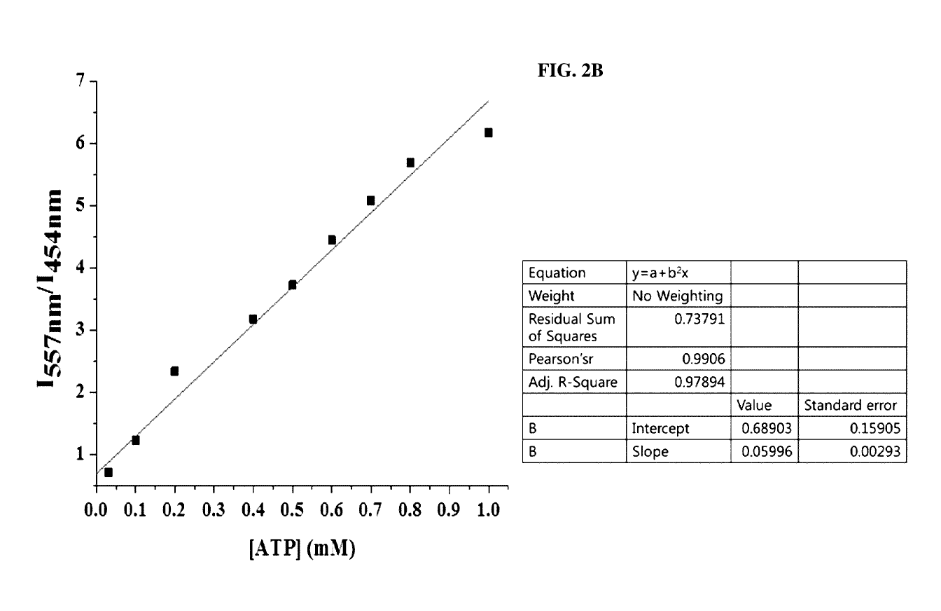

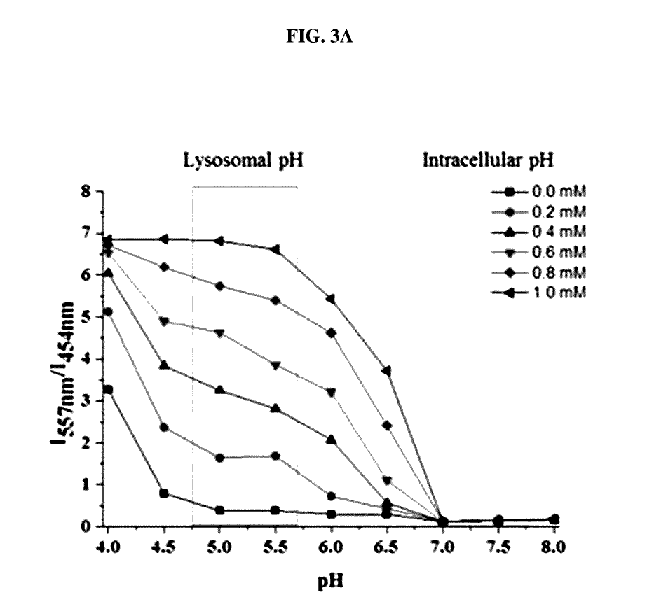

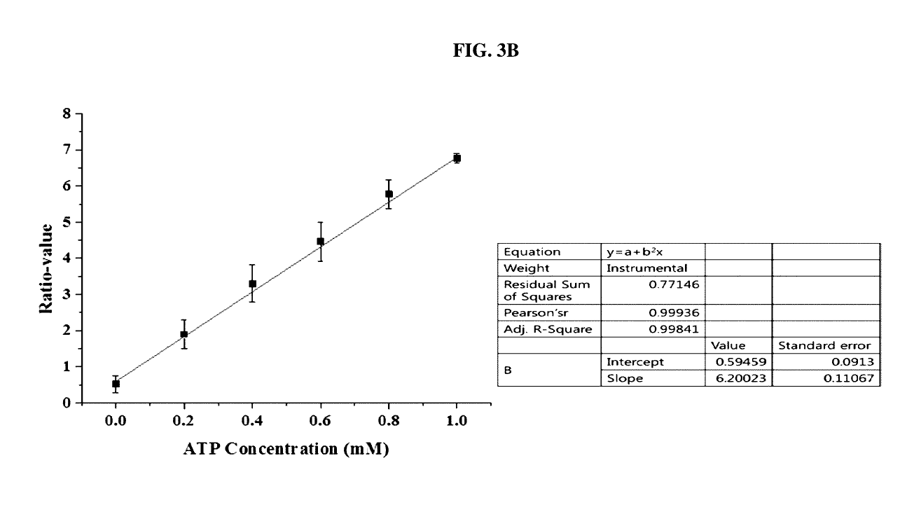

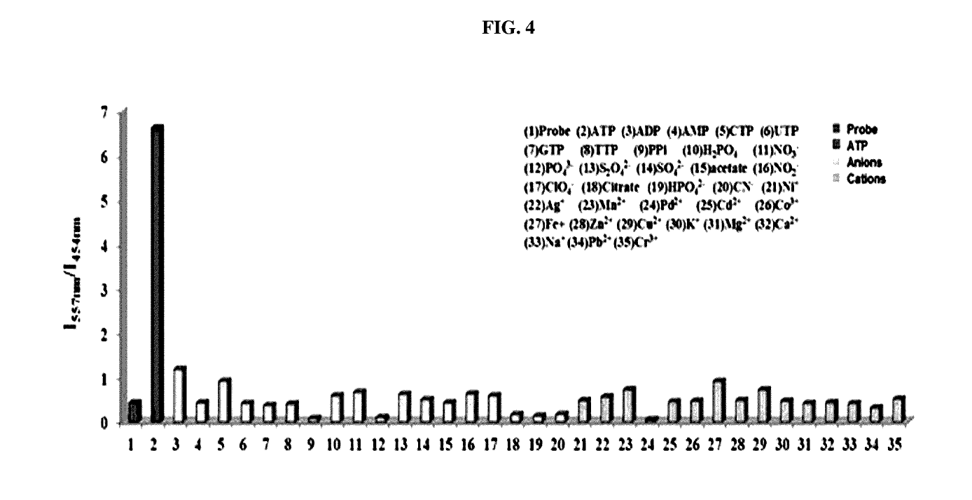

The present inventors observed the fluorescence characteristic of Compound 4 (R₁═R₂═H), which is the fluorescent probe according to embodiments of the present invention, and the results are provided in FIG. 2, FIG. 3, and FIG. 4.

Specifically, to observe the ATP detection characteristic of Compound 4, the present inventors measured a fluorescence spectrum (the fluorescence spectrum was obtained using a Photon Technical International Fluorescence System) by filling a quartz fluorescence cell (114F-QS, Hellma Analytics) with a 1 cm light path with a pH 5.5 PBS solution (with 0.1% acetonitrile) comprising 10 μM Compound 4 and ATP (0.0-1.0 mM), and graphs of the normalized fluorescence intensity (y-axis) with respect to the wavelength (x-axis) are shown in FIG. 2A. In FIG. 2A, the fluorescent probe detected ATP as the ATP concentration increased, and the fluorescence intensity greatly increased near 550 nm. In addition, to assess the feasibility of quantification by a ratiometric detection system, the correlation between the ATP concentration (x-axis) and the fluorescence ratio (the ratio between two types of fluorescence) (y-axis) was provided in FIG. 2B. As appears in FIG. 2B, the proportional relationship between the ATP concentration and the ratio between two types of fluorescence proves the feasibility of quantification by a ratiometric detection system.

In order to assess the correlation between the ATP detection by Compound 4 and the pH of the solution, the present inventors measured a fluorescence spectrum (the fluorescence spectrum was obtained using a Photon Technical International Fluorescence System) by filling a quartz fluorescence cell (114F-QS, Hellma Analytics) with a 1 cm light path with a PBS solution (with 0.1% acetonitrile) with a pH range of 4.0 to 8.0 comprising 10 μM Compound 4 and ATP (0.0-1.0 mM), and graphs of the fluorescence ratio (y-axis) with respect to the pH (x-axis) are shown in FIG. 3A. According to FIG. 3A, the detectability changed with pH. It was observed that ATP was detected only under acidic conditions (pH 4.8-5.8) of lysosomes, which are the only organelles maintaining acidic conditions in cells, and was not detected at near-neutral pH (pH 7.0-7.5), which corresponds to the pH of most cells. In addition, the fluorescence ratio (y-axis) that changes with respect to the ATP concentration (x-axis) is provided for the pH range of 4.5 to 5.5 in FIG. 3B. As appears in FIG. 3B, it was found that the ATP concentration could be quantified with an error range of ±0.12 mM within the mentioned pH range, based on the ratio between two types of fluorescence.

In order to examine the selectivity of Compound 4 in ATP detection, the present inventors measured a fluorescence spectrum (the fluorescence spectrum was obtained using a Photon Technical International Fluorescence System) by filling a quartz fluorescence cell (114F-QS, Hellma Analytics) with a 1 cm light path with a pH 5.5 PBS solution (with 0.1% acetonitrile) comprising Compound 4 at 10 μM and various cations and anions (ATP, ADP, AMP, cytidine triphosphate (CTP), uridine triphosphate (UTP), guanosine triphosphate (GTP), thymine triphosphate (TTP), inorganic phosphate (PPi), phosphoric acid (H₃PO₄), nitrate anions (NO₃⁻), phosphate anions (PO₄³⁻), dithionite ion (S₂O₄²⁻), sulfate ion (SO₄²⁻), acetate ion, nitrite ion (NO₂⁻), perchlorate ions (ClO₄⁻), citrate ions, monohydrogen phosphate ions (HPO₄²⁻), cyanide ions (CN⁻), nickel ions (Ni⁺), silver ions (Ag⁺), manganese ions (Mn²⁺), palladium ions (Pd²⁺), cadmium ions (Cd²⁺), cobalt ions (Co³⁺), iron ions (Fe⁺), zinc ion (Zn²⁺), copper ion (Cu²⁺), potassium ion (K⁺), magnesium ion (Mg²⁺), calcium ions (Ca²⁺), sodium ions (Na⁺), lead ions (Pb²⁺), and chromium ions (Cr³⁺)) at 1 mM, and the fluorescence ratios (y-axis) for various analytes (x-axis) were provided in FIG. 4. As shown in FIG. 4, the fluorescent probe (Compound 4) was found to be highly selective to ATP among various cations and anions.

### EXAMPLE 5

Assessment of Cytotoxicity of Fluorescent Probe

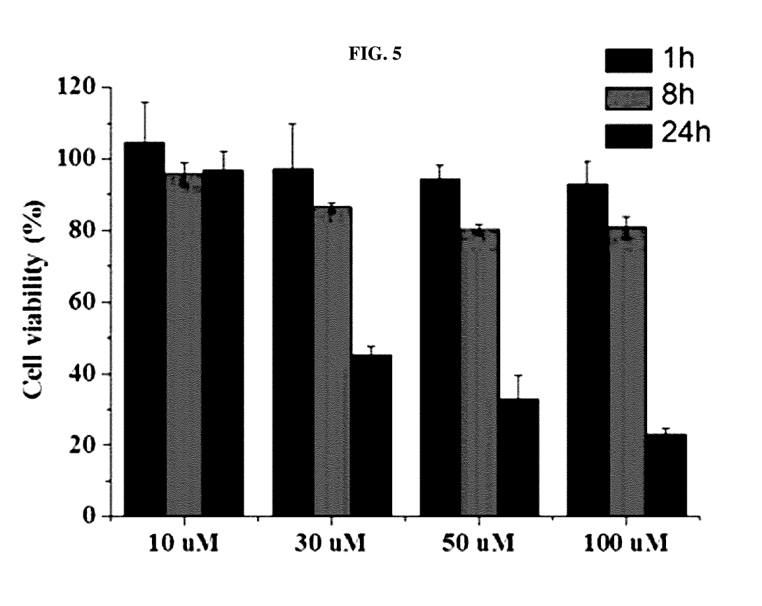

The present inventors examine the cytotoxicity of Compound 4 (R₁═R₂═H), which is the fluorescent probe according to embodiments of the present invention, by treating HeLa cells (human uterine cancer cells) with the compound, and the results are provided in FIG. 5.

To examine the cytotoxicity of Compound 4, an MTT assay was conducted on the HeLa cells and the cell viability was assessed.

Specifically, the cells (100 μL/well) were prepared in a 96-well plate at the density of about 5×10³ cells per well. The cells were treated with Compound 4 at the concentration of 10, 30, 50, or 100 μM, cultured for 1 hour, and then washed with a PBS buffer solution. 25 μL of a 5 mg/mL 3-(4,5-dimethyldiazol-2-yl)-2,5-diphenyltetrazolium bromide (MTT) solution was put in each well. After culturing for 2 hours at 37° C., the medium was removed, formazan crystals were dissolved in dimethyl sulfoxide (DMSO), and the absorbance was measured using a plate reader. According to FIG. 5, Compound 4 was confirmed to have low cytotoxicity up to the concentration of 10 μM within 24 hours (about 8% of the cells died). In addition, even when the concentration of Compound 4 was increased to 100 μM, it was confirmed that cytotoxicity within 8 hours was low (about 20% of the cells died), which indicated the possibility of conducting experiments in the corresponding range.

Therefore, it was found that the fluorescent probe according to embodiments of the present invention (Compound 4) was appropriate for obtaining a two-photon fluorescence microscopic image in a cell or a tissue and an animal model.

### EXAMPLE 6

Assessment of Lysosomal Selectivity of Fluorescence Probe

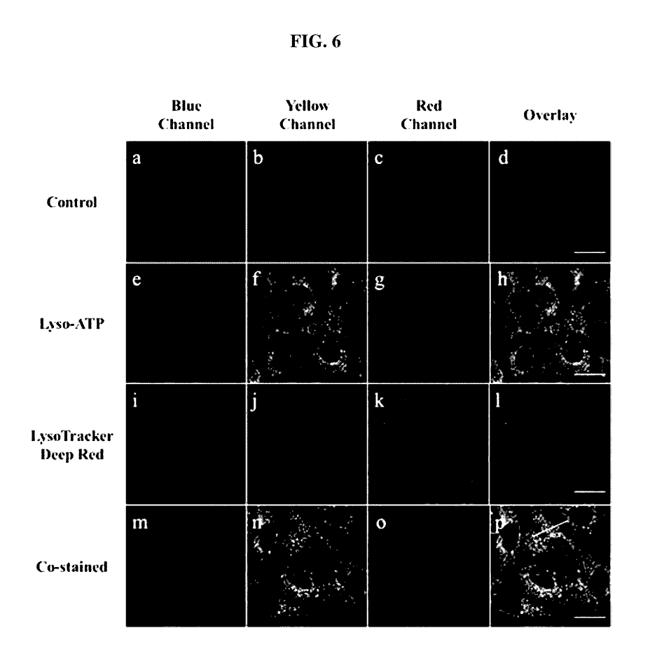

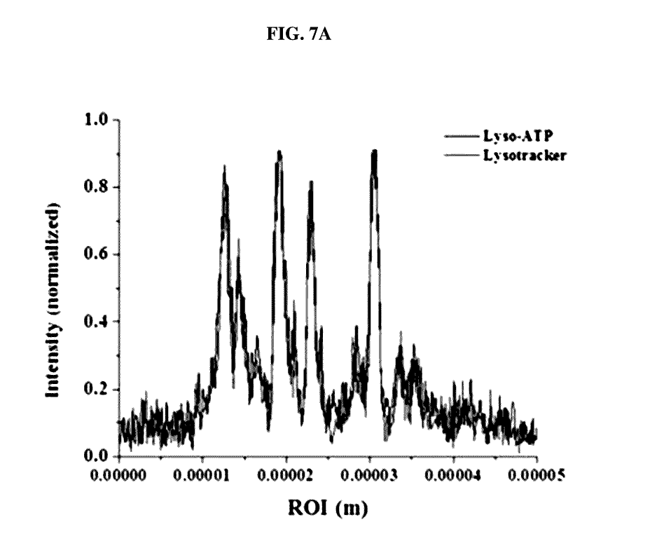

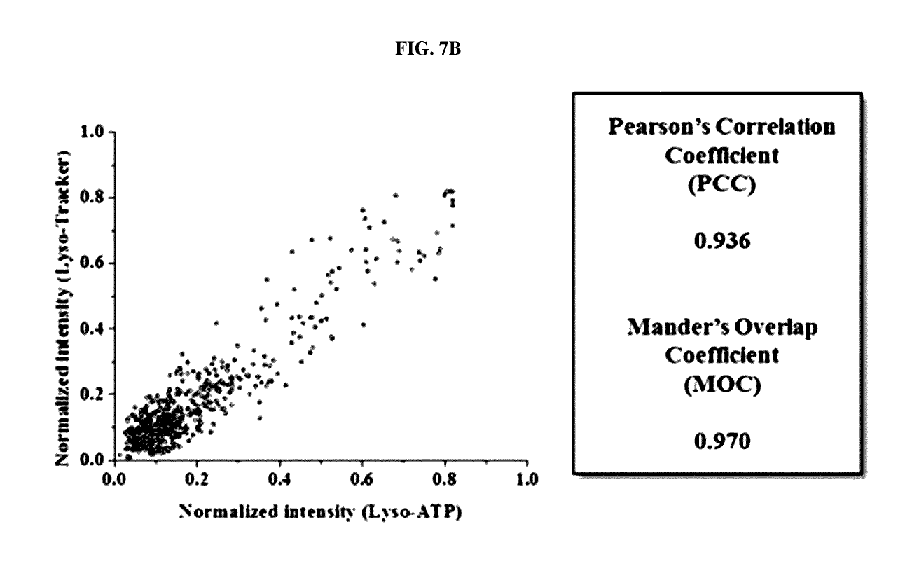

The present inventors treated HeLa cells with Compound 4 (R₁═R₂═H), which is a fluorescent probe, examined a fluorescence emission phenomenon through two-photon fluorescence microscopy, and assessed lysosomal selectivity by treating the cells also with LysoTracker (Life Technologies Corp.), which is a lysosome marker widely used to assess lysosomal selectivity, and then performing one-photon and two-photon fluorescence microscopy. The results are provided in FIG. 6 and FIG. 7. The scale bar in FIG. 6 is 25 μm.

Specifically, HeLa cells were treated with Compound 4, and then a fluorescence image thereof was observed with a two-photon fluorescence microscope (TCS SP5 II, Leica, Germany). Each cell was cultured in Minimum Essential Medium with Earle's Balanced Salts (MEM/EBSS) of Hyclone™ GE Healthcare Life Sciences and a Dulbecco's modified Eagle's medium (DMEM), and the culture was carried out at 37° C. The cultured cells were again cultured in a 96-well plate at 37° C. for 24 hours so that about 1×10⁵ cells were produced. Later, the cells were treated with 10 μM Compound 4 and then cultured for an additional hour. Then, the cell culture medium was removed from the cells, and the cells were washed three times with a PBS buffer solution and were fixed by introducing a 4% formic acid solution. The cells prepared as thus were observed through two-photon fluorescence microscopy to obtain fluorescence images, and the two-photon fluorescence microscopic images were obtained at a 780 nm excitation wavelength and through a laser power of 4 mW respectively in the blue channel (440-490 nm), yellow channel (550-600 nm), and red channel (650-700 nm). The results are provided in FIG. 6e, FIG. 6f, FIG. 6g, and FIG. 6h. As shown in FIG. 6e, information on the distribution of Compound 4 within a cell was obtained in the blue channel, and, as appears in FIG. 6f, information on the distribution of lysosomal ATP, which was detected by Compound 4, within a cell was obtained in the yellow channel.

The experiment was repeated with LysoTracker Deep Red (Life Technologies Corp.). HeLa cells were treated with LysoTracker, and then a fluorescence image thereof was obtained with a one-photon fluorescence microscope (TCS SP5 II, Leica, Germany). Each cell was cultured in Minimum Essential Medium with Earle's Balanced Salts (MEM/EBSS) of Hyclone™ GE Healthcare Life Sciences and a Dulbecco's modified Eagle's medium (DMEM), and the culture was carried out at 37° C. The cultured cells were again cultured in a 96-well plate at 37° C. for 24 hours so that about 1×10⁵ cells were produced. Later, the cells were treated with 50 nM LysoTracker and then cultured for an additional hour. Then, the cell culture medium was removed from the cells, and the cells were washed three times with a PBS buffer solution and were fixed by introducing a 4% formic acid solution. The cells prepared as thus were observed through one-photon fluorescence microscopy to obtain fluorescence images, and the one-photon fluorescence microscopic images were obtained at a 688 nm excitation wavelength and respectively in the blue channel (440-490 nm), yellow channel (550-600 nm), and red channel (650-700 nm). The results are provided in FIG. 6i, FIG. 6j, FIG. 6k, and FIG. 6l. As shown in FIG. 6k, information on the distribution of LysoTracker within a cell was obtained in the red channel, indicating the distribution of lysosomes within a cell. In addition, through the two individually conducted experiments, it was found that the imaging of Compound 4 and LysoTracker was possible through separate channels.

The lysosomal selectivity of Compound 4 in a cell was determined by the degree by which the distribution of lysosomal ATP detected by Compound 4 matched the distribution of lysosomes stained with LysoTracker. HeLa cells were simultaneously treated with Compound 4 and LysoTracker, and then a fluorescence image thereof was obtained with a one-photon and two-photon fluorescence microscope (TCS SP5 II, Leica, Germany). Each cell was cultured in Minimum Essential Medium with Earle's Balanced Salts (MEM/EBSS) of Hyclone™ GE Healthcare Life Sciences and a Dulbecco's modified Eagle's medium (DMEM), and the culture was carried out at 37° C. The cultured cells were again cultured in a 96-well plate at 37° C. for 24 hours so that about 1×10⁵ cells were produced. Later, the cells were treated with 10 μM Compound 4 and 50 nM LysoTracker and then cultured for an additional hour. Then, the cell culture medium was removed from the cells, and the cells were washed three times with a PBS buffer solution and were fixed by introducing a 4% formic acid solution. The cells prepared as thus were observed through one-photon and two-photon fluorescence microscopy to obtain fluorescence images, and the two-photon fluorescence was obtained at a 780 nm excitation wavelength and respectively in the blue channel (440-490 nm) and the yellow channel (550-600 nm), and the one-photon fluorescence microscopic image was obtained at a 688 nm excitation wavelength and in the red channel (650-700 nm). The results are provided in FIG. 6m, FIG. 6n, FIG. 6o, and FIG. 6p. FIG. 6m and FIG. 6n show Compound 4 observed through two-photon fluorescence microscopy, and FIG. 6o shows LysoTracker observed through one-photon fluorescence microscopy. By comparing FIG. 6n and FIG. 6o, the distribution of lysosomal ATP detected by Compound 4 could be compared with the distribution of lysosomes stained with LysoTracker, and, as shown in FIG. 6p, the distributions matched to a considerable extent.

In order to quantify the degree by which the distributions in FIG. 6n and FIG. 6o matched each other, a linear region of interest (ROI) was set (FIG. 6p) and the patterns within the region were compared to each other, and the graphs of the fluorescence intensity (y-axis) with respect to the ROI (x-axis) are shown in FIG. 7A. According to the results shown in FIG. 7A, the appearance of the fluorescence intensity of Compound 4 in a blue line and the fluorescence intensity of LysoTracker in a red line bear much similarity to each other. In addition, as appears in FIG. 7B, the colocalization factor, which is a number corresponding to the degree by which the two fluorescence intensities match in appearance, was found to be as much as about 0.95, which indicated that the distribution of lysosomal ATP detected by Compound 4 matched the distribution of lysosomes stained with LysoTracker.

Therefore, it was found that the fluorescent probe according to embodiments of the present invention (Compound 4) was highly selective to lysosomes.

### EXAMPLE 7

Observation of Two-Photon Microscopic Image of Rat Tissue Treated with Fluorescent Probe: Assessment of ATP Selectivity of Fluorescent Probe

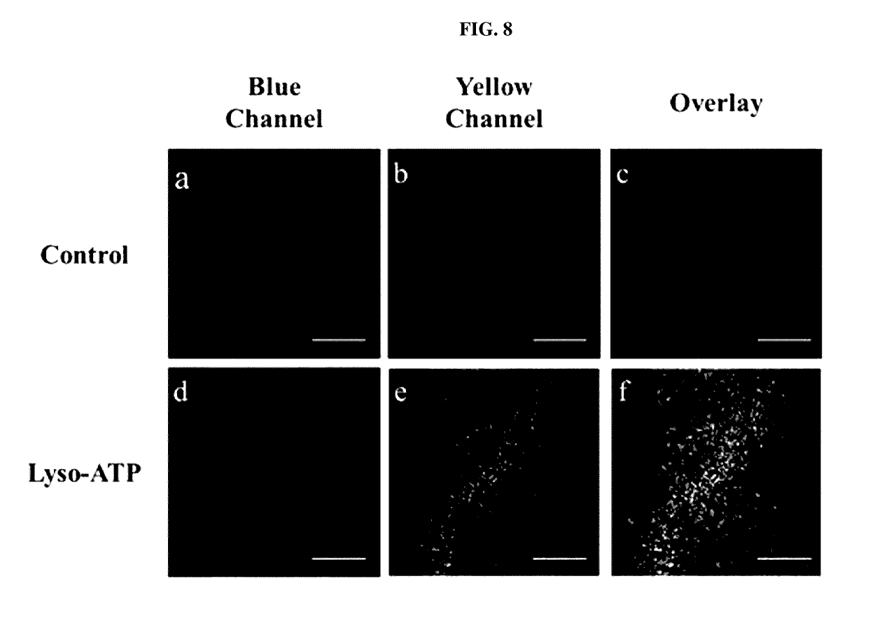

The present inventors treated a rat brain tissue with Compound 4 (R₁═R₂═H), which is the fluorescent probe according to embodiments of the present invention and observed the changes in fluorescence through two-photon fluorescence microscopy, and the results are provided in FIG. 8. The scale bar in FIG. 8 is 80 μm.

Specifically, to obtain a two-photon fluorescence microscopic image of a rat tissue treated with Compound 4, an experiment was carried out under a condition of isolated from light (in a dark room) using a C57BL6 rat (5 weeks old, male, Samtako Inc.). The brain of the rat was dissected and washed with a PBS buffer solution, and then the individual organs were frozen with dry ice for 5 minutes. The frozen organs were broken with a hammer, and 50 μm-thick tissue slice samples were prepared using a cryostat machine (CM3000, Leica). To fixate an organ to the cryostat machine, an optical cutting temperature compound ((OCT; 10% polyvinyl alcohol, 25% polyethylene glycol, and 85.5% inactive species) was used. Each tissue slice sample was placed on a specimen block (Paul Marienfeld GmbH & Co. KG), the specimen block was immersed in 4% paraformaldehyde for 10 minutes and then was washed with a PBS buffer solution, and the tissue was again fixated using a mounting medium (Gel Mount™, Biomeda Corp.). The prepared tissue slice sample was immersed in a PBS buffer solution comprising 30 μM Compound 4 for 20 minutes, washed three times with a PBS buffer solution, and then fixed with 4% paraformaldehyde. The fluorescence was observed through two-photon fluorescence microscopy. The two-photon fluorescence microscope was configured with a 20× objective lens (HCX APO L 20×/1.00 W, Leica, Germany), and the observation was carried out using a Ti: Sapphire laser (Chameleon Ultra II, Coherent) with 6 mW laser output and at a 780 nm two-photon excitation wavelength. According to the results shown in FIG. 8, it was found that Compound 4 selectively detected lysosomal ATP even in a deep tissue through two-photon fluorescence microscopy.

Embodiments of the invention are described above, and it will be understood by those skilled in the art that various modifications can be made without departing from the scope of the present invention and without changing essential features. Therefore, the above-described examples should be considered in a descriptive sense only and not for the purposes of limitation.

What is claimed is:

1. A compound represented by Structural Formula 1 below: 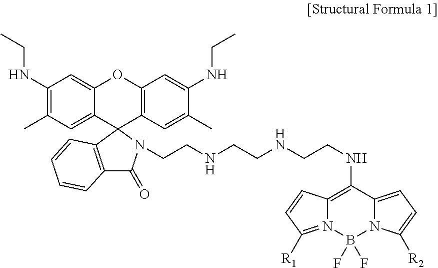
where in the Structural Formula 1,
R₁ and R₂ are each independently hydrogen (H), a methyl (Me) group, an allyl group, or a C₂-C₁₂ alkyl group.

2. The compound of claim 1, wherein the compound represented by Structural Formula 1 is 10-((2-((2-((2-(3′,6′-bis(ethylamino)-2′,7′-dimethyl-3-oxospiro[isoindoline-1,9′-xanthen]-2-yl)ethyl)amino)ethyl)amino)ethyl)amino)-5,5-difluoro-5H-dipyrrolo[1,2-c:2′,1′-f][1,3,2]diazaborinin-4-ium-5-uide).

3. A method of detecting adenosine triphosphate (ATP), the method comprising:
treating a cell or tissue with a composition comprising the compound of claim 1 or a pharmaceutically acceptable salt of the compound.

4. The method of claim 3, wherein the method selectively detects ATP in a lysosome.

5. The method of claim 3, wherein the composition increases a fluorescence intensity in a wavelength range of 520 to 580 nm by binding with ATP.

6. The method of claim 3, wherein the method detects ATP in a pH range of 4.5 to 6.

7. A method of preparing a compound represented by Structural Formula 1 below, the method comprising:
a) adding triethylene tetramine to rhodamine 6G ((E)-ethyl 2-(6-(ethylamino)-3-(ethylimino)-2,7-dimethyl-3H-xanthene-9-yl)benzoate) to synthesize 2-(2-((2-((2-aminoethyl)amino)ethyl)amino)ethyl)-3′,6′-bis(ethylamino)-2′,7′-dimethylspiro[isoindoline-1,9′-xanthen]-3-one, which is a rhodamine 6G derivative compound; and
b) reacting the 2-(2-((2-((2-aminoethyl)amino)ethyl)amino)ethyl)-3′,6′-bis(ethylamino)-2′,7′-dimethylspiro[isoindoline-1,9′-xanthen]-3-one with a compound represented by Structural Formula 2 below in dichloromethane (DCM) to synthesize the compound represented by Structural Formula 1 below.

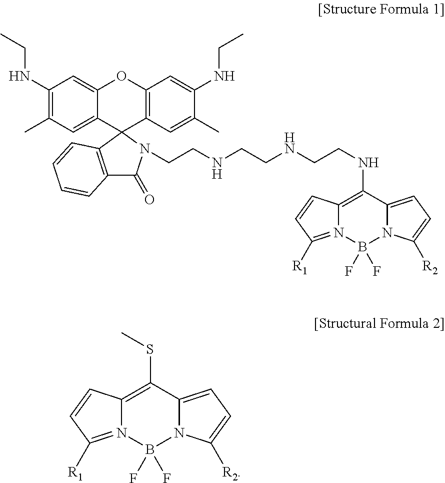

8. A method of imaging a cell or a tissue, the method comprising:
a) treating a cell or tissue with the compound of claim 1 or a pharmaceutically acceptable salt thereof; and
b) observing, through one-photon or two-photon fluorescence microscopy, a fluorescence emitted from the cell or tissue due to lysosomal ATP.

9. A method of quantifying lysosomal ATP, the method comprising:
a) treating a cell or tissue with the compound of claim 1 or a pharmaceutically acceptable salt thereof;
b) measuring a fluorescence intensity from the cell or tissue at wavelengths ranging from 440 to 460 nm and from 520 to 580 nm; and
c) quantifying ATP concentration by calculating a ratio between the two measured fluorescence intensities.
